(19) United States
(12) Patent Application Publication
(10) Pub. No.: US 2013/0253306 A1
(43) Pub. Date: Sep. 26, 2013

(54) SYSTEM, METHOD AND FACILITY FOR REDUCING PEDIATRIC PATIENT ANXIETY DURING MEDICAL DIAGNOSTIC IMAGING PROCEDURES

(71) Applicant: General Electric Company, Schenectady, NY (US)

(72) Inventor: Douglas P. Dietz, Pewaukee, WI (US)

(73) Assignee: General Electric Company, Schenectady, NY (US)

(21) Appl. No.: 13/895,116

(22) Filed: May 15, 2013

Related U.S. Application Data
(63) Continuation of application No. 12/428,835, filed on Apr. 23, 2009.
(60) Provisional application No. 61/154,272, filed on Feb. 20, 2009.

(51) Int. Cl. A61B 5/00; A61M 21/02; A61B 6/03

(52) U.S. Cl. CPC A61B 5/0046; A61B 6/03; A61M 21/02
USPC 600/411; 600/427; 29/594

(57) ABSTRACT

A facility for performing a medical examination includes a room, a medical imaging system located in the room, and a plurality of elements installed on at least one of the room and the medical imaging system, the elements collectively creating a first theme that forms a physical environment at least partially surrounding a patient, wherein the theme is presented in a manner to reduce the level of patient anxiety and fear experienced by the patient being imaged. A medical imaging system is also described. A method of imaging a patient using the facility, a method of remodeling an existing facility, and a method of determining a theme to be used in a medical facility are also described.

### CROSS REFERENCE TO RELATED APPLICATION

The present application is a continuation of and claims priority to U.S. patent application Ser. No. 12/428,835, filed Apr. 23, 2009, which claims priority to U.S. Provisional Application Ser. No. 61/154,272 filed Feb. 20, 2009, the disclosures of which are incorporated herein.

### BACKGROUND OF THE INVENTION

The present invention relates generally to medical imaging systems, and more particularly to a method and system for reducing the anxiety level of a patient, particularly a pediatric patient, before and during the procedure in order to further reduce image artifacts attributable to patient motion during image data capture.

Medical imaging systems scan patients to obtain medical information, and involve several imaging modalities, including: computed tomography (“CT”), positron emission tomography (“PET”), combined CT and PET (“PET-CT”), single photon emission computed tomography (“SPECT”), X-ray, gamma camera imaging, magnetic resonance (“MR”), and others, including the combinations or hybrids thereof.

Medical images constructed (or reconstructed) from data acquired by the medical imaging equipment and/or systems are deleteriously affected by patient motion during the data acquisition phase and this is a well known problem. Image quality must be above a certain acceptable threshold to be considered useful for diagnosis or treatment. In the event the image is unacceptable for use or diagnosis the patient must be scanned again and a second dose of an imaging agent drug must be administered. This inefficiency is not merely burdensome and increasingly more traumatic for the child, it is a poor use of hospital resources, equipment and staff.

In order to reduce patient motion during an imaging procedure, the imaging system operator typically instructs the patient to remain substantially motionless while the imaging information is being acquired. However, involuntary motion encountered in medical imaging systems is also common and a source of image artifacts, and may be caused by numerous physiological parameters. For example, breathing motion, motion of the heart, e.g. accelerated heart rate, and/or motion involuntary motion of a head, arm, or leg of the patient. Increased patient anxiety is known to cause increases in some of the patient's involuntary physiological parameters such as the patients breathing or the patient's heart rate, resulting in increased patient movement during imaging.

Other patient motions that are not involuntary and yet not readily controllable by the patient are movements are particularly pronounced with the pediatric patient (i.e., children). This pediatric patient anxiety is often attributable to the intimidating look and overall appearance of the imaging system as viewed through the child's eyes regardless of the modality. Typically the imaging system is installed in a dedicated imaging room within a hospital or imaging center. In most of the rooms where the imaging equipment is installed, there are a variety of additional support systems and subsystems such as computers, telephones, general medical care devices such as blood pressure cuffs, furniture, equipment consoles, etc., which is just about anything supportive of the procedure or medical care in general.

While both the conventional imaging system and the conventional imaging system room are designed to improve the performance of the imaging system and to enhance the operation of the system by the medical personnel, the clinical appearance and smell, the unfamiliar shape and large size of the equipment, and associated operational sounds of it, etc., are known to cause anxiety and fear in pediatric patients undergoing the medical imaging procedure. In turn, the anxiety and fear exhibited by the child is manifested in both voluntary and involuntary motion including, but not limited to, elevated heart rates, rapid breathing, fidgeting, trembling, and nervousness resulting in unwanted body movement during image data capture.

To counteract voluntary and involuntary patient motion, physicians commonly administer sedatives to children to cause physical and mental relaxation of the child patient thereby minimizing patient motion enabling a better scan. In the pediatric setting, the sedation process itself also adds an element of fear and anxiety. While sedative injections or inhalants used for children are common in imaging procedures they are wholly undesirable. Wherever sedation is required, it is preferred to utilize the minimum required sedative to calm the patient, minimize patient motion attributed to anxiety and fear, etc., and perform the accurate imaging. The physician chooses the dosage of sedative to offset the patient's anxiety level.

Scientific studies have proven when pediatric patients are distracted from or desensitized to the conventional clinical imaging environment, the need for sedation noticeably and appreciably decreases while image quality, accuracy and efficiency improves.

Therefore, a need exists for a facility, an imaging system and a method to reduce pediatric patient anxiety and fear associated with medical imaging procedures in order to improve scan success and image quality while minimizing the use of sedatives.

### BRIEF DESCRIPTION OF THE INVENTION

In one embodiment, a facility for performing a medical imaging procedure includes a room having a medical imaging system located in the room, and a plurality of inventive thematic elements installed on the room walls, ceiling, floor, and the medical imaging system. The thematic elements collectively create a theme that forms a physical environment and includes the imaging equipment that at least partially surrounds the patient, wherein the theme is presented in a manner to reduce the level of patient anxiety and fear experienced by the child/patient being imaged.

In another embodiment, a medical imaging system is provided. The medical imaging system includes a medical imaging device, an imaging table adapted to support and/or move the patient with respect to the medical imaging device, and a plurality of thematic elements installed on the medical imaging device including the table. The thematic elements collectively creating a theme that forms a physical environment with the imaging room at least partially surrounding a patient, wherein the theme is presented in a manner to reduce the level of patient anxiety and fear experienced by the patient being imaged.

In another embodiment, a method of remodeling an existing medical imaging facility to reduce patient anxiety is provided. The method includes selecting a room in the medical imaging facility, determining a theme to be installed in the selected room, and modifying the selected room based on the determined theme, wherein the selected theme is presented in a manner to reduce the level of patient anxiety and fear experienced by the patient being imaged.

The present invention can be described as follow: a medical imaging facility constructed according to a theme to reduce the anxiety level of a patient, comprising at least one room having a plurality of walls and a ceiling; a medical imaging system operably positioned within the room; a first plurality of thematic elements affixed to the room; a second plurality of thematic elements affixed to the medical imaging system; wherein the first plurality of thematic elements and second plurality of thematic elements are pre-selected to cooperatively create a theme that forms a physical environment for the room, such that the environment partially surrounds a patient, wherein the theme is presented in a manner to reduce the level of patient anxiety and fear experienced by the patient being imaged.

The present invention may also be described as follows: a medical imaging system comprising a medical imaging unit; an imaging table adapted to move a medical patient with respect to the medical imaging unit; and a plurality of thematic elements installed on the medical imaging unit and the table, the elements collectively creating a theme that forms a physical environment at least partially surrounding a patient, wherein the theme is presented in a manner to reduce the level of patient anxiety and fear experienced by the patient being imaged.

The inventive medical imaging system can be described as including a computed tomography (CT) imaging unit, a magnetic resonance imaging (MRI) unit, a positron emission tomography (PET) imaging unit, an ultrasound imaging unit, and an X-ray imaging unit, and any combination thereof.

The present invention may also be described to include: a method of remodeling an existing medical imaging facility in a manner to reduce the level of patient anxiety and fear experienced by the patient being imaged, said method comprises selecting a room in the medical imaging facility having a medical imaging system in it; selecting a visual theme to be installed in the selected room; and modifying the selected room and the appearance of the medical imaging system by installing the selected theme inside the room and on the medical imaging system, wherein the selected theme is presented in a manner to reduce the level of patient anxiety and fear experienced by the patient being imaged.

The invention may further include a first plurality of thematic elements and a second plurality of thematic elements configured to collectively represent a first theme having an appearance of a non-medical environment such as a rain forest, space travel, railroad, sporting activity, circus, zoo, or just about any morally suitable theme imaginable. The plurality of thematic elements may also partially comprise a plurality of thematic appliqués associated with the theme, at least one paint scheme, or an audio-visual display.

An inventive method associated with the present invention may also include adapting a medical imaging room, facility or system to reduce the anxiety and fear level of a patient being imaged, by determining an age of a plurality of potential patients; selecting a theme based on the determined age, wherein the theme is presented in a manner to reduce an anxiety level of the patient being imaged; and installing a plurality of elements on at least one of a medical imaging room and a medical imaging system installed in the room, the elements collectively creating the selected theme and forming a physical environment at least partially surrounding the patient.

The descriptions set forth above are exemplary and not intended to be limiting, and combinations or alternate embodiments thereof are considered to be within the scope of the present invention.

### BRIEF DESCRIPTION OF THE DRAWINGS

FIG. 1 is a top view of an exemplary medical imaging facility in accordance with an embodiment of the present invention.

FIG. 2 is a perspective view of an exemplary medical imaging system that may be used with the facility shown in FIG. 1 in accordance with an embodiment of the present invention.

FIG. 3 is a perspective view of an exemplary imaging system room that includes an exemplary theme in accordance with an embodiment of the present invention.

FIG. 4 is a perspective view of another exemplary imaging system room that includes another exemplary theme in accordance with an embodiment of the present invention.

FIG. 5 is a flowchart illustrating an exemplary method in accordance with an embodiment of the present invention.

FIG. 6 is a flowchart illustrating an exemplary method in accordance with an embodiment of the present invention.

FIG. 7 is a flowchart illustrating an exemplary method in accordance with an embodiment of the present invention.

FIG. 8 is an exemplary storyboard in accordance with an embodiment of the present invention.

### DETAILED DESCRIPTION OF THE INVENTION

The foregoing summary and detailed description of certain embodiments of the present invention will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

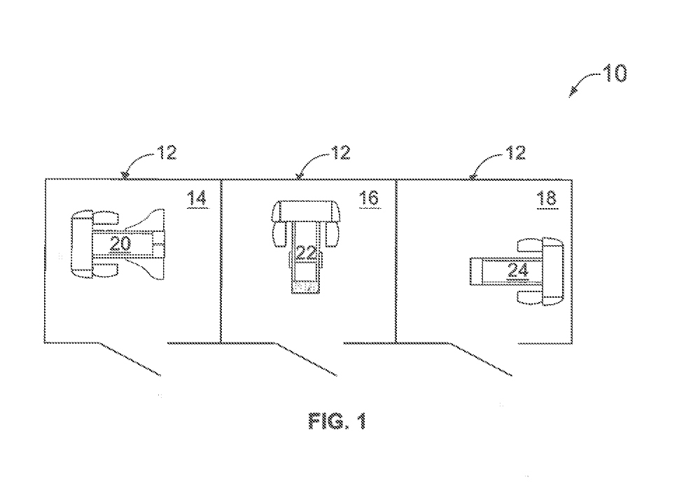

FIG. 1 is a top view of an exemplary medical imaging facility 10 in accordance with an embodiment of the present invention. The medical imaging facility 10 includes at least one medical imaging room designated generally by the reference numerals 12, and may include a plurality of medical imaging rooms 12. For example, facility 10 may include a first medical imaging room 14, a second medical imaging room 16, and a third medical imaging room 18. In the exemplary embodiment, the medical imaging facility 10 is a dedicated imaging center or hospital. For example, the medical imaging facility may be embodied as any structure that is configured to perform medical imaging, such as an office building that includes a suite that is configured to perform medical imaging or a mobile medical imaging vehicle.

In the exemplary embodiment, each of the medical imaging rooms includes a medical imaging system. For example, the first medical imaging room 14 includes a medical imaging system 20, the second medical imaging room 16 includes a medical imaging system 22, and the third medical imaging room 18 includes a medical imaging system 24. The medical imaging systems 20, 22 and 24 may be embodied as a computed tomography (CT) imaging system, a magnetic resonance imaging (MRI) system, a positron emission tomography (PET) imaging system, an ultrasound imaging system, an X-ray imaging system, or a single photon emission computed tomography (SPECT) imaging system. Each medical imaging room 12 may include a single medical imaging system or multiple medical imaging systems, and may include a multi-modality imaging system that includes at least two of the medical imaging modalities described above.

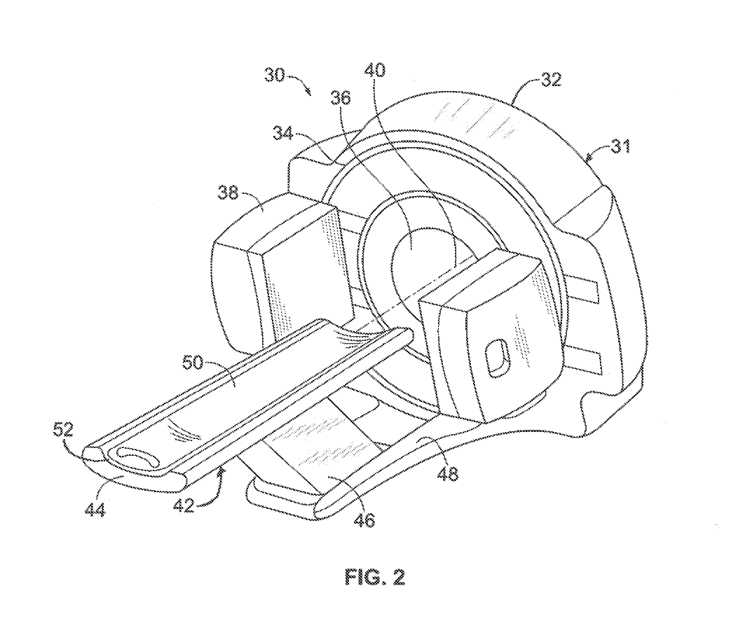

FIG. 2 is a pictorial view of an exemplary medical imaging system 30 (numerals 20, 22, and 24 of FIG. 1). In the exemplary embodiment, the medical imaging system 30 in this view is a nuclear medicine imaging system, and more particularly, a single photon emission computed tomography (SPECT) imaging system. The medical imaging system 30 includes an integrated medical imaging unit 31 that includes a gantry 32 that further includes a rotor 34 oriented about a gantry central bore 36. The rotor 34 is configured to support one or more nuclear medicine (NM) cameras 38 (two cameras 38 are shown), such as, but not limited to gamma cameras, SPECT detectors, multi-layer pixelated cameras (e.g., Compton camera) and/or PET detectors. It should be noted that when the medical imaging system 30 includes a CT camera or an x-ray camera, the medical imaging system 30 also includes an x-ray tube (not shown) for emitting x-ray radiation towards the detectors. In various embodiments, the cameras 38 are formed from pixelated detectors, and the rotor 34 is further configured to rotate axially about an examination axis 40.

The medical imaging system 30 also includes an imaging table 42 that is adapted to move a medical patient (not shown) with respect to the medical imaging unit 31. The imaging table 42 may include a bed 44 slidingly coupled to a bed support system 46, which may be coupled directly to a floor or may be coupled to the gantry 32 through a base 48 coupled to the gantry 32. The bed 44 may include a stretcher 50 slidingly coupled to an upper surface 52 of the bed 44. The imaging table 42 is configured to facilitate ingress and egress of a patient (not shown) into an examination position that is substantially aligned with the examination axis 40. During an imaging scan, the imaging table 42 may be controlled to move the bed 44 and/or stretcher 50 axially into and out of the bore 36. The operation and control of the imaging system 10 may be performed in any manner known in the art. It should be noted that the various embodiments may be implemented in connection with imaging systems that include rotating gantries or stationary gantries.

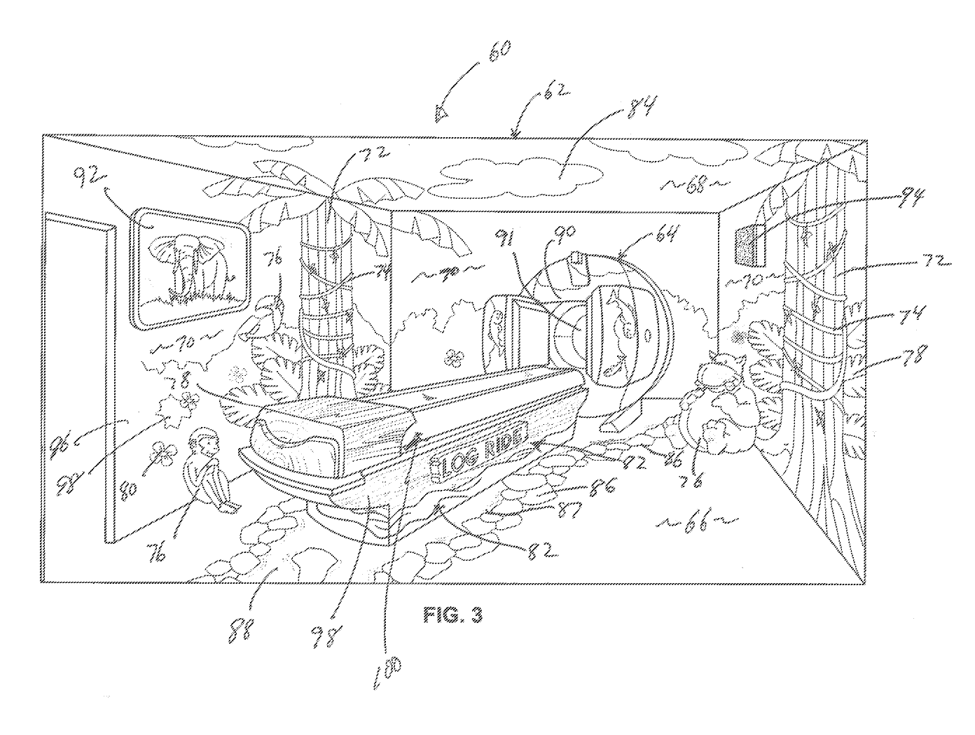

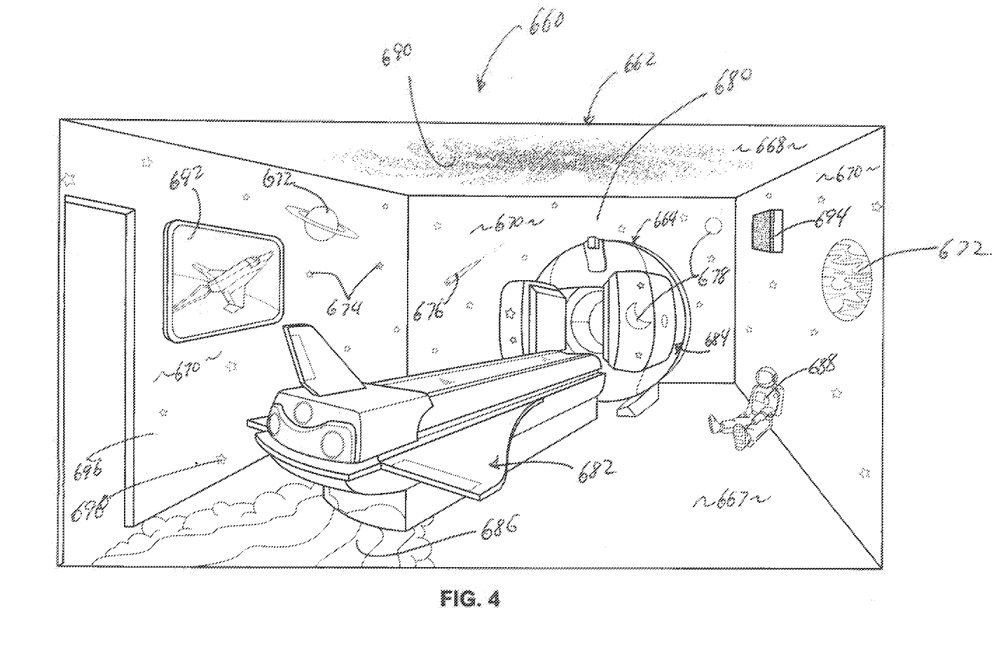

With reference to FIGS. 3 and 4, two themes designated generally by the reference numerals 60 and 660 respectively may be used in a medical imaging environment. As discussed above, other themes (not shown) may also be utilized. In the exemplary embodiment, the themes are designed to reduce the level of patient anxiety and fear experienced by the patient being imaged, and preferably the pediatric child patient by merging the visual appearance of the imaging equipment with the friendly appearance of the room surroundings.

More specifically, as discussed herein, conventional imaging rooms and the conventional imaging system in it may cause a patient to experience an unpleasant emotion or fear in anticipation of the medical examination or procedure and while this is particularly common in young children, the present invention has been shown to minimize fear and anxiety of the patient overall, as well as to reduce the need for sedation in children. To facilitate a reduction in the patient's fear and anxiety, and thus improve the image quality and potentially reduce the quantity of sedative delivered to a patient, the conventional medical imaging room is modified with a theme, e.g., 60 and 660.

FIGS. 3 and 4 depict exemplary medical imaging rooms, designated generally by the reference numerals 62 and 662, respectively. The medical imaging rooms 62 and 662 include an exemplary medical imaging system, such as the medical imaging system 64 and 664 resting on the floor 66 and 667, a ceiling 68 and 668, a plurality of walls 70 and 670, and a door 72, and 672 (FIGS. 3 and 4 respectively). In the exemplary embodiment, the medical imaging rooms 62 and 662 also include a theme that forms a physical environment at least partially surrounding a patient.

A “theme”, as used herein, represents a unifying idea or subject that is implemented in the medical imaging room, including thematic elements associated with and affixed to the medical imaging equipment. For example, some exemplary themes may include a rainforest and log ride, an outer space travel theme, a jungle theme, a sports theme, a child's birthday party, a doll house, a cartoon character theme, a pirate adventure, a safari theme, a circus theme, fantasy related themes, a museum theme, a farmyard theme, railroad theme and/or a fairytale theme, to name a few. Moreover, the theme may be related to a special character or medical imaging system mascot that is designed to be used in a medical image environment to reduce a patient's anxiety level. It should be realized that the above-described themes are exemplary and that the medical imaging room may be configured to represent other additional themes not discussed above and yet having the same function and possesses the required enablement.

All themes, those shown as 60 and 660 as well as those not shown and considered part of the present invention and comprises alternate embodiments thereof, are configured to reduce the level of patient anxiety and fear experienced by the patient being imaged by changing the conventional appearance and character of an imaging room to a theme unrelated to clinical and medical applications and procedures, thereby improving the image quality and clinical reliability, and reducing the need for sedation of the child.

In FIGS. 3 and 4, the exemplary embodiment having themes 60 and 660 use a plurality of thematic elements. With respect to FIG. 3, the thematic elements include, for example, a tree 72, a vine 74, animals 76, plants 78, flowers 80, a log ride 82, clouds 84, river stones 86, river 88, waterfall 90 (only partially visible, with the remainder inside and behind the bore 91), video display 92, and audio device 94. With respect to FIG. 4, the thematic elements include, planets 672, stars, 674, comet 676, moons 678, open space 680, space ship 682, lunar module 684, rocket exhaust trail 686, spaceman 688, galaxy, 690, video display 692, and audio device 694. The foregoing elements represent physical modifications made to both the medical imaging room and the medical imaging system. For example, some physical modifications will be made to each, i.e., the medical imaging rooms 62 and 662 (FIGS. 3 and 4 respectively) and the medical imaging systems 64 and 664 to comprise a contiguous theme selected. As such, the combination of the thematic elements enable the child patient to have fun thereby allowing the patient to relax for better image quality and efficiency, as well as avoid or minimize the need for sedatives.

Referring again to FIGS. 3 and 4, one of the thematic elements may be embodied as a scenic design that is implemented using, in the preferred embodiment, a combined painting or painted on scheme 96, 696, and appliqué scheme 98, 698, having a plurality of colors that is applied to at least a portion of the medical imaging room 62 and 662 respectively. Appliqué as used herein represents a decorative image or design that is fabricated as a unitary element and then affixed to a surface of the medical imaging room 62 and 662 and necessarily to a surface of the medical imaging system 64 and 664. The appliqués are particularly useful for the equipment associated with the medical imaging system 64 and 664 because they are preferably removable and do not interfere with the operation of the equipment or the approved status as medical devices.

Scenic design, as used herein represents an overall background “picture” that provides a physical appearance that represents the selected theme as produced in part by the applied painting designs and the foreground overlaid onto the painting scheme by the appliqué. In addition, only a portion of the walls 70 and 670 need to be painted as an appliqué overlays a portion to give a three dimensional appearance. For example, in the exemplary embodiment, the theme illustrated in FIG. 3 is a “Rain Forest Theme” and theme represented in FIG. 4 is a space travel theme. As such, the combination of the thematic elements of FIG. 3 represent a Rain Forest and Log Ride 82 (with indicia) using appliqués having a wooden appearance applied to the imaging table, designated generally by the reference numeral 100, while the thematic elements of FIG. 4 depict a space travel theme complete with space vehicle 682 and both themes present a contiguous complimentary cooperating visual appearance between the medical imaging system and the room.

Scenic designs may be installed by substantially permanently by painting at least a portion of the medical imaging room and applying an appliqué. Optionally, a scenic design may be installed by utilizing a portable scenic device such as a picture or video display 92 and 692 (FIGS. 3 and 4) that is mounted on a wall of the medical imaging room. It should be realized, that when either the permanent scene design or the portable scenic device is used, the scenic design or the scenic device should substantially cover the entire wall and the imaging equipment to maintain the overall theme of the medical imaging rooms 62 and 662.

In the exemplary embodiment, shown in FIG. 3, the river 88 is surrounded by a plurality of stones 86 and a background color that represents dirt or sand 87. In one embodiment, the first scenic design 80 is installed by applying a paint mixture to the floor 66. Optionally, the first scenic design 80 may be formed using a combination of paint and semi-permanent fixtures. For example, a semi-permanent fixture appliqué, representing the river 88 and stones 86 may then be placed on the sand colored floor 66. Additionally, the same type of complementary cooperating scheme(s) is used for the wall and the thematic elements attached thereto as well as the imaging equipment to represent the inventive contiguous theme(s).

More specifically, in one embodiment, the tree appliqué 72 and/or the bird appliqué 76 may be fabricated as a sticker, or adhesive panel or label, that depicts a representation of a tree and a bird, respectively. In another embodiment, at least one of the tree or bird appliqués 72 and 76 may be fabricated as three-dimensional structure that is then affixed to a wall 70. Moreover, the tree appliqué 72 may be fabricated such that at least a portion of it extends at least partially outward from the wall.

In the exemplary embodiment, the appliqués are easily removable to facilitate either relocating the appliqués or removing them altogether to install another theme.

As discussed above, another one of the thematic elements may be embodied as a full-sized display device 92 and 692 (FIGS. 3 and 4) that is configured to display a portion of the overall theme 60 and 660. Display, as used herein represents an electronic device that presents information or images in visual form. Similarly, another one of the thematic elements may be embodied as an audio system 94 and 694 that is configured to transmit sounds related to the themes. For example, the sounds may include rain, wind, or various other noises that enhance the themes, and cooperate with the actual sound of the equipment thereby resembling the roar of a waterfall 90 (FIG. 90), or rocket 682 travel sounds (FIG. 4). In addition, a supplemental lighting system (not shown) may be included to enhance the experience.

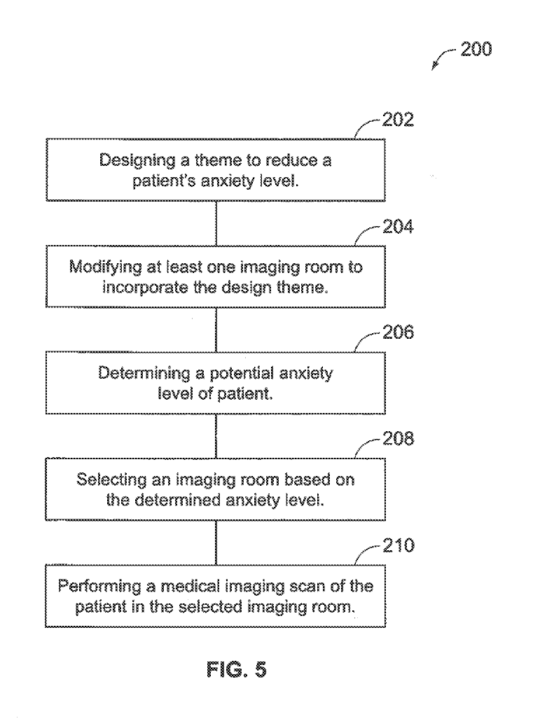

FIG. 5 is a flowchart illustrating an exemplary method 200 for imaging a patient using a medical imaging system constricted to reduce patient anxiety and fear for better image quality and scan efficiency.

At 202, a theme is designed to reduce the patient's anxiety, and thus reduce patient movement during the imaging process and improve the image quality as a result of the decreased movement. A theme, as used herein, represents a unifying idea or subject that is implemented in the medical imaging room to reduce patient anxiety.

At 204, at least one imaging room is modified by applying a plurality of elements on at least a portion of the imaging room and the medical imaging system, the elements collectively associated with the selected theme. For example, the walls of the imaging room may be painted to incorporate a scenic design associated with the selected theme. Appliques, props, and indicia may then be installed in the room.

At 206, a potential anxiety level of a patient is determined. In one embodiment, the patient's anxiety level may be determined based on a plurality of physiological parameters of the patient. For example, the anxiety level may be determined based on the patient's vital signs such as a patient's heart rate or breathing rate, or example. The current heart and/or breathing rate may then be compared to previous heart rate or breathing rate data acquired from the same patient. Patient's experiencing an increased heart or breathing rate is deemed to have an increased anxiety level. In another embodiment, the potential anxiety level of a patient is determined based on the patient's a priori knowledge of the medical imaging procedure. For example, one patient may have no knowledge of the imaging procedure and therefore have increased anxiety. Whereas another patient who has been previously imaged may have little or no anxiety. In one embodiment, the patient's anxiety is based on the patient's age. For example, a young patient may have no knowledge of the imaging procedure and therefore have increased anxiety. Whereas an older patient who has been previously imaged may have little or no anxiety.

At 208, an imaging room is selected based on the potential anxiety level. For example, assuming that the child is young or female and has relatively high anxiety level, an operator may determine that an imaging room having the rainforest theme, shown in FIG. 3, is best suited to reduce the anxiety level of the young female patient. Whereas an older male may be best suited for the outer space theme shown in FIG. 4. Moreover, the patient's age may also be used to identify the imaging room. For example, a young patient may be imaged in the imaging room having a more youthful appeal, such as the rainforest scene. Whereas, an older patient may prefer the imaging room having an older appeal such as the outer space theme or a sports theme for example.

At 210 a medical imaging scan of the patient is performed in the selected imaging room. As discussed above, the medical scan may be performed using a computed tomography (CT) imaging system, a magnetic resonance imaging (MRI) system, a positron emission tomography (PET) imaging system, an ultrasound imaging system, an X-ray imaging system, and/or a single photon emission computed tomography (SPECT) imaging system.

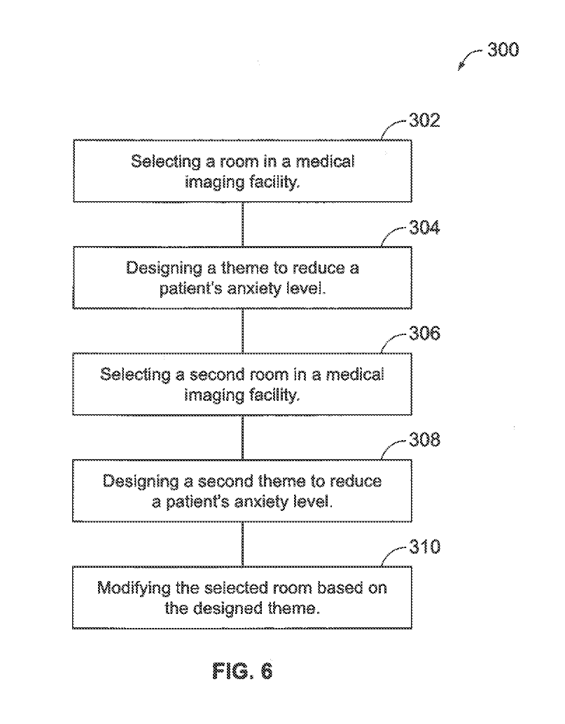

FIG. 6 is a flowchart illustrating an exemplary method 300 of remodeling an existing medical imaging facility to reduce patient anxiety.

At 302, a room in the medical imaging facility is selected. In the exemplary embodiment, the room is a medical imaging room that includes a medical imaging system, such as one of the medical imaging systems described herein.

At 304, a theme is designed to be installed in the selected room. As discussed above, the theme is designed based on a plurality of factors, some of the factors include a patient's age and a patient's gender. Other factors may include the quantity of patient's falling within a predetermined age group of gender. For example, assuming that eighty percent of the patients being imaged are relatively young, the theme may be designed having a more youthful appeal.

At 306, a second room in the medical imaging facility is selected. In the exemplary embodiment, the second room is a medical imaging room that includes a medical imaging system, such as one of the medical imaging systems described herein.

At 308, a second different theme is designed to be installed in the second room. As discussed above, the theme is designed based on a plurality of factors, some of the factors include a patient's age and a patient's gender. Other factors may include the quantity of patient's falling within a predetermined age group of gender. For example, assuming that twenty percent of the patients being imaged are adults, the second theme may be designed having a more adult appeal.

At 310, at least one of the first and second rooms is modified using one of the designed themes. More specifically, at least one of the first and second rooms is modified by applying a plurality of elements on at least a portion of the imaging room and the medical imaging system, the elements collectively associated with the selected theme. For example, the walls of the imaging room may be painted to incorporate a scenic design associated with the selected theme. Appliques, props, and indicia may then be installed in the room. In the exemplary embodiment, the first imaging room is modified to include a first theme and the second imaging room is modified to include a different second theme. During operation, each of the selected themes is configured to reduce an anxiety level of a patient being imaged in the room.

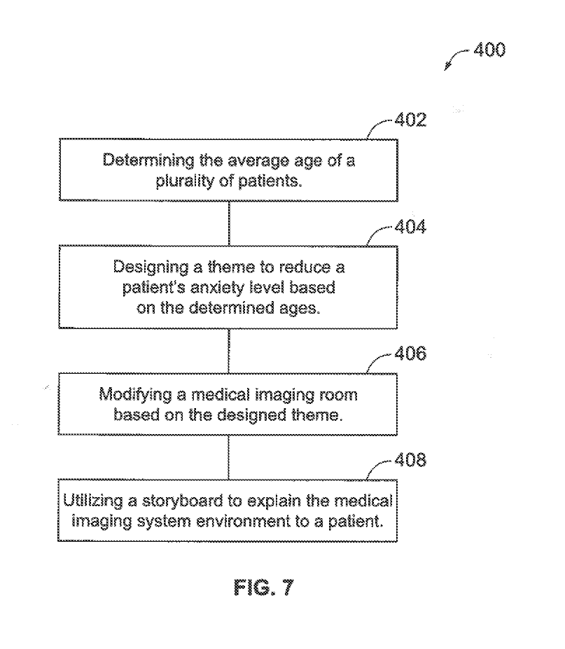

FIG. 7 is a flowchart illustrating an exemplary method 400 of producing a medical imaging system environment that is adapted to reduce the anxiety level of a patient.

At 402, an age of a plurality of potential patients is determined.

At 404, a theme is designed based on the determined age. The theme is configured to reduce an anxiety level of a patient being image. More specifically, a theme is designed to reduce the patient's anxiety, and thus reduce patient movement during the imaging process and improve the image quality as a result of the decreased movement. A theme, as used herein, represents a unifying idea or subject that is implemented in the medical imaging room to reduce patient anxiety. For example, assuming that the patient is a child, a more youthful theme is designed. Whereas, another design may be directed to an adult. It should be realized that numerous themes may be designed and implemented in numerous respective imaging rooms. In this manner, a specific theme may be designed that facilitates reducing the anxiety level in young children and adults of both genders.

At 406, at least one imaging room is modified by applying a plurality of elements on at least a portion of the imaging room and the medical imaging system, the elements collectively associated with the selected theme. For example, the walls of the imaging room may be painted to incorporate a scenic design associated with the selected theme. Appliques, props, and indicia may then be installed in the room. Additionally, displays, sound systems, and audio systems may be employed as part of the overall theme. In the exemplary embodiment the installed theme includes a plurality of elements that collectively create the selected theme and forms a physical environment at least partially surrounding a patient being imaged.

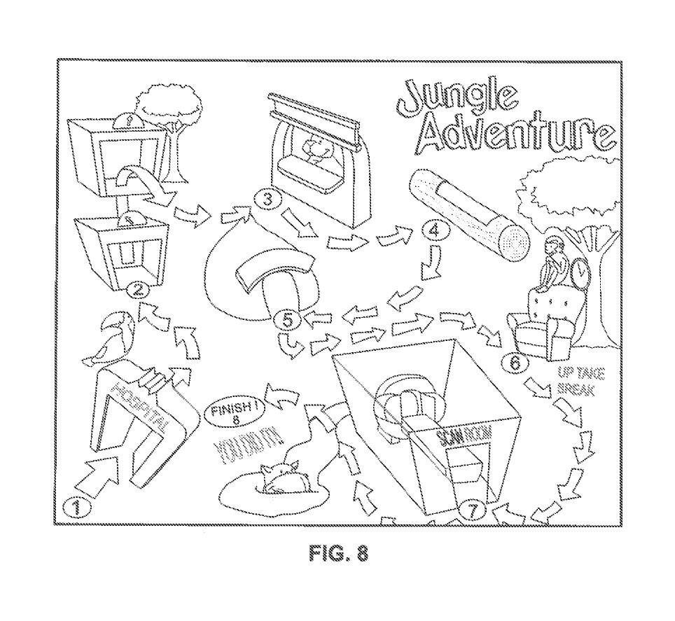

At 408, a storyboard is utilized to explain the medical imaging system environment to a patient. Referring to FIG. 8, an exemplary storyboard 420 is shown. In the exemplary embodiment, the storyboard 420 represents a simplified illustration of the medical imaging environment that may be utilized to explain the medical imaging system environment to a child. For example, as discussed above, children often exhibit increased levels of anxiety caused both by their unfamiliarity with the imaging environment in general and also their unfamiliarity with the medical imaging equipment. As such, the storyboard 420 may be utilized by an adult to explain the environment to a child and thus reduce the child's anxiety level prior to the child being imaged. For example, the storyboard 420 indicates that the expected route and procedures to which the child will encounter prior to the child entering the imaging room 12. As a result, the child becomes familiar with the overall environment and is thus more relaxed when entering the medical imaging room 12.

Described herein are a medical facility, a medical imaging system, and methods that are configured to reduce the patient's anxiety prior to the patient being imaged. A technical effect of the systems and methods described herein is reducing the amount of sedation to a patient and also increasing image quality. More specifically, as discussed above, the stark imaging environment combined with the patient's unfamiliarity with the imaging environment often increases the patient's anxiety, especially in children. As such, when utilizing a conventional imaging system, the patient's movement caused by the anxiety may extend the time required to perform the imaging, create a backlog at the imaging room, and/or decrease the quality of the images produced. To relax the patient, a doctor may increase the quantity of sedation given to the patient. While the sedation levels are within acceptable medical guidelines, the increased sedation may also result in an increase in the amount of time required for the patient to recover from sedation. As a result, the time required for a patient to being imaged in a conventional imaging system room is further increased.

The exemplary embodiments described herein reduce the time required to perform patient imaging, increase the quality of the images, and also reduces the amount of sedative given to a patient. Specifically, the imaging rooms described herein include themes that are specifically designed to reduce the anxiety level in patient's including younger patients. The imaging system room includes a theme that represents a place that is readily apparent to the child being imaged. The themes are implemented using a plurality of theme-related elements. The theme-related elements may include scenic designs on the walls and the imaging system itself Appliques, indicia, and props may also be placed in the imaging room to further enhance and define the selected theme. The themes described herein are readily apparent to a child being imaging and therefore immediately reduces the child's anxiety level before imaging. As a result, the patient may require a reduced amount of sedative than is used to image a child in a conventional imaging system, thereby decreasing the time required to perform imaging and improving image quality. Additionally, some patients, e.g. children, may not require anesthesia thus eliminating the need to inject the patient with a topical anesthetic which also leads to increased anxiety. Moreover, because of the patient has a reduced anxiety level, and thus moves less during the scan, the quantity of scans is reduced and the requirement to perform repeat scans is reduced. Additionally, the quantity of delays caused by waiting for a patient to recover from anesthesia is reduced, thus increasing the patient throughput through the imaging system room.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

What is claimed is:

1. A medical imaging facility comprising:
a medical imaging unit positioned in an imaging room;
an imaging table adapted to move a medical patient with respect to the medical imaging unit; and
a plurality of thematic elements installed on the medical imaging unit and the table, the plurality of thematic elements collectively creating a theme that forms a physical environment at least partially surrounding a patient, wherein the theme is presented in a manner to reduce the level of patient anxiety and fear experienced by the patient being imaged;
wherein the theme includes an audible component, the audible component utilizing an inherent sound of the medical imaging unit is incorporated therein.

2. The medical imaging facility of claim 1 comprising an audio system positioned within the imaging room and configured to transmit sounds related to the theme that cooperate with the actual sound of the equipment.

3. The medical imaging facility of claim 2 wherein the audio system is configured to enhance the theme by cooperating with the sound of the equipment to resemble one of a roar of a waterfall and a rocket.

4. The medical imaging facility of claim 1 wherein the plurality of thematic elements is partially comprised of a plurality of thematic appliqués and a thematic painted on scheme cooperatively configured to produce the theme.

5. The medical imaging facility of claim 4 wherein at least one of the plurality of thematic appliqués is fabricated such that at least a portion of it extends at least partially outward from a wall of the room, and wherein the plurality of thematic appliqués are each fabricated as unitary elements and affixed respectively to the plurality of walls and to the medical imaging unit.

6. The medical imaging facility of claim 1 comprising an electronic display positioned within the imaging room and adapted to display a moving picture associated with the theme.

7. The medical imaging facility of claim 1 wherein the plurality of thematic elements includes a three-dimensional structure affixed to a wall of the imaging room.

8. The medical imaging facility of claim 1 wherein one of the plurality of thematic elements comprises a plurality of removable appliqués.

9. The medical imaging facility of claim 1 wherein the plurality of thematic elements includes a semi-permanent appliqué placed on a floor of the imaging room.

10. The medical imaging facility of claim 1 wherein the plurality of thematic elements include a first set of thematic elements installed on the medical imaging unit and the table, and a second set of thematic elements affixed to the wall of the imaging room.

11. The medical imaging facility of claim 10 wherein the first set of thematic elements and the second set of thematic elements include one or more elements that are common to both.

12. The medical imaging facility of claim 10 wherein the first set of thematic elements and the second set of thematic elements include one or more elements that collectively create the theme that represents a unifying subject.

13. The medical imaging facility of claim 10 wherein the first set of thematic elements and the second set of thematic elements include one or more elements that collectively create a visual theme having elements that are all common to the visual theme.

14. The medical imaging facility of claim 1 wherein the plurality of thematic elements comprises at least one stationary element associated with the theme and attached to the imaging unit, and protrudes from the imaging unit.

15. The medical imaging facility of claim 1 wherein the plurality of thematic elements include a non-medical three-dimensional structure associated with the theme that is positioned within the imaging room.

16. The medical imaging facility of claim 1 comprising a storyboard positioned proximate the medical imaging unit, the storyboard configured to provide a graphical representation of the imaging process to the patient being imaged, to include a graphical illustration of the expected route and procedures to which a child will encounter such that an adult can explain the environment to the child using the storyboard.

17. The medical imaging facility of claim 16 wherein the storyboard includes a visual representation of the medical imaging unit and the storyboard includes at least one thematic element that corresponds to the theme.

18. The medical imaging facility of claim 17 wherein at least one element on the storyboard that corresponds to the theme is an element that is also installed within the imaging room that corresponds to the theme.

19. A method of remodeling an existing medical imaging facility in a manner to reduce the level of patient anxiety and fear experienced by the patient being imaged, the method comprising:
selecting a room in the medical imaging facility having a medical imaging system in it, wherein the room includes a floor, a ceiling, and a plurality of walls formed between the floor and the ceiling;
selecting a visual theme to be installed in the selected room;
modifying the selected room and the appearance of the medical imaging system by installing elements of the visual theme on the walls and on the floor of the room, and on the medical imaging system, wherein the visual theme is presented in a manner to reduce the level of patient anxiety and fear experienced by the patient being imaged; and
installing an audio system within the room that is configured to transmit sounds that resemble sounds related to the theme.

20. The method of claim 19 configuring the audio system to transmit sounds that cooperate with an actual sound of the equipment

21. The method of claim 19 wherein modifying comprises installing elements related to the theme on the walls and on the medical imaging system that are common to both.

22. The method of claim 19 comprising installing an electronic display positioned within the imaging room that is adapted to display a moving picture associated with the theme.

23. The method of claim 19 comprising affixing a three-dimensional structure to a wall of the imaging room that is associated with the visual theme.

24. The method of claim 19 comprising configuring the audio system to transmit sounds that resemble one of a roar of a waterfall and a rocket that corresponds to the theme.

25. The method of claim 19 wherein modifying further comprises applying a first painting scheme to at least one of the ceiling and the floor, and applying a second different painting scheme to at least one of the plurality of walls, the first and second painting schemes each forming a portion of the selected theme.

26. A medical imaging facility constructed according to a theme to reduce the anxiety level of a patient, comprising:
at least one room having a plurality of walls and a ceiling;
a medical imaging system operably positioned within the room;
a first plurality of elements associated with a theme and affixed to the room;
a second plurality of thematic elements associated with the theme and affixed to the medical imaging system; and
an audio system positioned within the at least one room that is configured to transmit sounds related to the theme, wherein the transmitted sounds are selected to cooperate with the operation of the equipment.

27. The medical imaging facility of claim 26 wherein the first plurality of elements and the second plurality of elements include one or more elements that are common to both the first plurality and the second plurality of elements, wherein the first and second plurality of elements are pre-selected to cooperatively create a theme that forms a physical environment for the room, such that the environment partially surrounds a patient, wherein the theme is presented in a manner to reduce the level of patient anxiety and fear experienced by the patient being imaged.

28. The medical imaging facility of claim 26 comprising an electronic display positioned within the at least one room and adapted to display a moving picture associated with the theme.
